SEQUENCE LISTING (2 sequences)

SEQ ID NO: 1 - protein, 153 aa, Equus caballus
GLSDGEWQQVLNVWGKVEADIAGHGQEVLIRLFTGHPETLEKFDEFKHLKTEAEMKASEDLKKHGTVVLTALGGILKKKGHHEAELKPLAQSHATKHKIPIKYLEFISDAIIHVLHSKHPGDFGADAQGAMTKALELFRNDIAAKYKELGFQG

SEQ ID NO: 2 - protein, 259 aa, Bos taurus
SHHWGYGKHNGPEHWHKDFPIANGERQSPVDIDTKAVVQDPALKPLALVYGEATSRRMVNNGHSFNVEYDDSQDKAVLKDGPLTGTYRLVQFHFHWGSSDDQGSEHTVDRKKYAAELHLVHWNTKYGDFGTAAQQPDGLAVVGVFLKVGDANPALQKVLDALDSIKTKGKSTDFPNFDPGSLLPNVLDYWTYPGSLTTPPLLESVTWIVLKEPISVSSQQMLKFRTLNFNAEGEPELLMLANWRPAQPLKNRQVRGFPK

(19) United States
(12) Patent Application Publication
(10) Pub. No.: US 2010/0035291 A1
(43) Pub. Date: Feb. 11, 2010

(54) METHOD FOR DEGRADING PEPTIDES, METHOD FOR ANALYZING PEPTIDES, DEVICE FOR DEGRADING PEPTIDES AND DEVICE FOR ANALYZING PEPTIDES

(75) Inventors: Kenji MIYAZAKI, Minato-ku (JP); Yo Tabuse, Minato-ku (JP); Hiroko Someya, Minato-ku (JP); Wataru Hattori, Minato-ku (JP); Hisao Kawaura, Minato-ku (JP)

Correspondence Address: SUGHRUE MION, PLLC, 2100 PENNSYLVANIA AVENUE, N.W., SUITE 800, WASHINGTON, DC, 20037, US

(73) Assignee: NEC Corporation, Tokyo (JP)

(21) Appl. No.: 12/507,440

(22) Filed: Jul. 22, 2009

(30) Foreign Application Priority Data
Jul. 24, 2008 (JP) 2008-191432

(51) Int. Cl. C12Q 1/37; C12N 9/50; C12M 1/00; G01N 27/00

(52) U.S. Cl. 435/23; 435/219; 435/287.1; 204/600

(57) ABSTRACT

A fragmenting reaction of peptide is achieved while maintaining the isolated state of peptide. Isolated peptide fractions isolated by electrophoresis are prepared in flow paths. Subsequently, prepared peptide fractions are dried by each of the flow paths. Then, dried peptide fractions are in contact with protease. Then, independent liquid membranes of a solvent are formed over the surfaces of peptide fractions, which have been in contact with protease, disposed on the flow paths, respectively.

This application is based upon and claims the benefit of priority from Japanese patent application No. 2008-191432, filed on Jul. 24, 2008, the disclosure of which is incorporated herein in its entirety by reference.

### BACKGROUND

1. Technical Field

The present invention relates to a method for degrading peptides, a method for analyzing peptides utilizing the method for degrading peptides, a device for degrading peptides, and a device for analyzing of the peptide utilizing the device for degrading peptides.

2. Background Art

In recent years, according to significant developments in proteo-mix technologies, realizations of so-called tailor-made medical treatments are expected. The tailor-made medical treatment is a medical treatment, in which proteins/peptides appeared in an individual patient are examined, and information on post-transitional modifications, RNA processing and protein processing, which cannot be obtained from genome information are utilized to achieve analyses of states of diseases, thereby providing medical treatments and preventions for such diseases. In the present time, fundamental studies for realizing such tailor-made medical treatments are actively made.

Energetic studies are continued in the areas of analysis techniques for blood serum proteins among such fundamental studies. Besides, studies for integrating data related to form and/or quantity of protein obtained from actual samples with other diagnosis information to provide the conditions for mining data are continued. it is expected that blood contains proteins/peptides, which are capable of functioning as markers for diseases. In addition, diagnostic approaches by the analysis of serum proteins can be carried out by simply taking blood, and thus are advantageous in lower physical/mental strains of an individual patient.

Further, protein-separating apparatuses are developed for handling blood serum samples, which utilize ultratrace levels of samples to achieve rapid separations with high separability. Quantifications of individual proteins/peptides separated and purified by such devices and assignments of quantified data to a database are becoming critical.

Analysis of serum protein is generally conducted by the following routine. First of all, individual protein and peptide separated and purified from a blood serum sample are fragmented. Subsequently, mass of each fragment is analyzed, and referencing of the result with the database is carried out. This allows assignments of protein and peptide.

Here, fragmentations of separated and purified proteins and the peptides are achieved by utilizing enzyme specific for amino acids (trypsin, lysyl endopeptidase, V8 protease and the like). Reactions for fragmentations are generally conducted within solutions or conducted in a condition for being maintained by a gel carrier.

Reference with database is achieved by matching measured molecular weight with theoretical molecular weight of fragment, which can be obtained from sequence information in the database. When a mass spectrometer is utilized, further fragmentation is conducted by exposing peptide fragment with a gas, and matching of the obtained further fragmented peptide with theoretical molecular weight obtained from sequences in the database. As such, actually measured molecular weight of peptide fragment is matched with sequence information of amino acids to provide enhanced probability of the assignments.

Currently, several proposals as approaches for analyzing protein and peptide are presented (for example, see WO 2005/078447 and Japanese Patent Laid-Open No. 2004-294431).

### SUMMARY

However, it is known that complicated sorting operations are required for fragmenting peptides while maintaining the separated state of peptides in related art. Therefore, labor and time are required for fragmentation reaction of peptides. In addition, problems of causing losses and contaminations of the samples in such operations are caused. Therefore, an establishment of a process for fragmenting peptides without causing a loss and/or a mixing of a sample and with less operation is expected.

An exemplary object of the invention is to provide a method for degrading peptides, which is capable of allowing fragmenting reaction of peptides while maintaining the separated state of peptides, a device for degrading peptides employing such method, and a method for analyzing peptides employing such method for degrading peptides, and a device for analyzing peptides employing such method.

A method for degrading peptide according to one exemplary aspect of the present invention includes preparing two or more isolated peptide fractions in carriers; drying the peptide fractions for each of the carriers; bringing a protease into contact with the dried peptide fractions; and forming independent liquid membranes of a solvent respectively on surfaces of the peptide fractions contacted with the protease for each of the carriers.

A method for analyzing peptide according to another exemplary aspect of the present invention includes a mass analysis of peptide fraction degraded by the above-described method for degrading peptide is conducted.

A device for degrading peptide according to further exemplary aspect of the present invention includes a sample holding unit for holding a carrier carrying dried mixed sample, the mixed sample containing two or more isolated peptide fraction and a protease, the protease being in contact with peptide fraction; a vapor supplying unit for supplying water vapor in the mixed sample; and a sensor for detecting formations of respectively independent liquid membranes of a solvent over the peptide fraction by the water vapor for each of the flow paths, the peptide fraction being in contact with the protease.

A device for analyzing peptide according to yet other exemplary aspect of the present invention includes a mass analysis section for conducting mass analysis of peptide fraction degraded in the above-described device for degrading peptide.

According to the present invention, a fragmenting reaction of peptide can be achieved while maintaining the isolated state of peptide.

### BRIEF DESCRIPTION OF THE DRAWINGS

The above and other objects, advantages and features of the present invention will be more apparent from the following description of certain exemplary embodiments taken in conjunction with the accompanying drawings, in which:

FIG. 1 is a flow chart, useful in describing a process for degrading peptides according to an exemplary embodiment;

FIG. 2 is a flow chart, useful in describing a method for analyzing peptides according to an exemplary embodiment;

FIG. 3 is a schematic diagram, illustrating a device for degrading peptide according to an exemplary embodiment;

FIG. 4 is a chart, showing a spectrum of fragmented peptide fragmented by trypsin derived from equine apomyoglobin;

FIG. 5 is a chart, showing a spectrum of fragmented peptide fragmented by trypsin derived from bovine carbonic anhydrase;

FIG. 6 is a schematic diagram of a protein-isolating chip;

FIGS. 7A and 7B are charts, showing results of scanning over flow paths for bovine carbonic anhydrase labeled with Cy3 fluorochrome condensed by employing a chip for isoelectric focusing, and more specifically, FIG. 7A shows a result measured after the freeze drying just after the electrophoresis, and FIG. 7B shows a result measured after the trypsin reaction;

FIG. 8 is a spectrum of a mass analysis of materials obtained from peptide fraction around the flow path position A10;

FIG. 9 is a spectrum of a mass analysis of materials obtained from peptide fraction around the flow path position A12;

FIG. 10 shows amino acid sequence of equine apomyoglobin and fragment obtained by breaking equine apomyoglobin with trypsin, and molecular weights thereof (MH⁺); and

FIG. 11 shows amino acid sequence of bovine carbonic anhydrase and fragment obtained by breaking bovine carbonic anhydrase with trypsin, and molecular weights thereof (MH⁺).

### EXEMPLARY EMBODIMENT

The invention will be now described herein with reference to illustrative exemplary embodiments. Those skilled in the art will recognize that many alternative exemplary embodiments can be accomplished using the teachings of the present invention and that the invention is not limited to the exemplary embodiments illustrated for explanatory purposed.

Exemplary implementations according to the present invention will be described in detail as follows in reference to the annexed figures. In all figures, an identical numeral is assigned to an element commonly appeared in the figures, and the detailed description thereof will not be repeated.

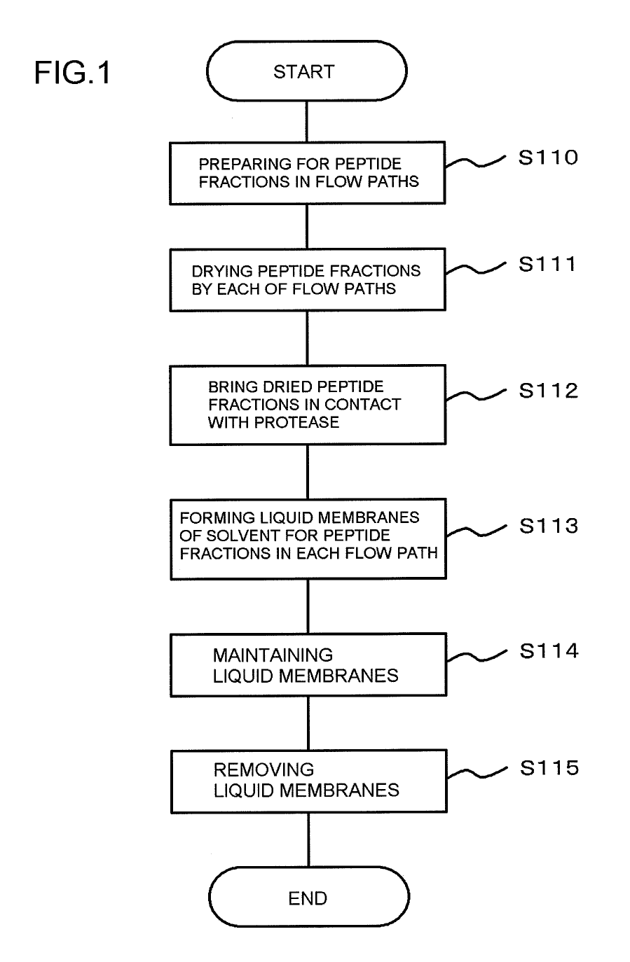

FIG. 1 is a flow chart for describing a method for degrading peptides according to the present exemplary embodiment. First of all, isolated peptide fractions are prepared in flow paths (S110). Subsequently, prepared peptide fractions are dried by each of the flow paths (S111). Then, dried peptide fractions are in contact with protease (S112). Then, independent liquid membranes of a solvent are formed over the surfaces of peptide fractions, which have been in contact with protease, disposed on the flow paths, respectively (S113).

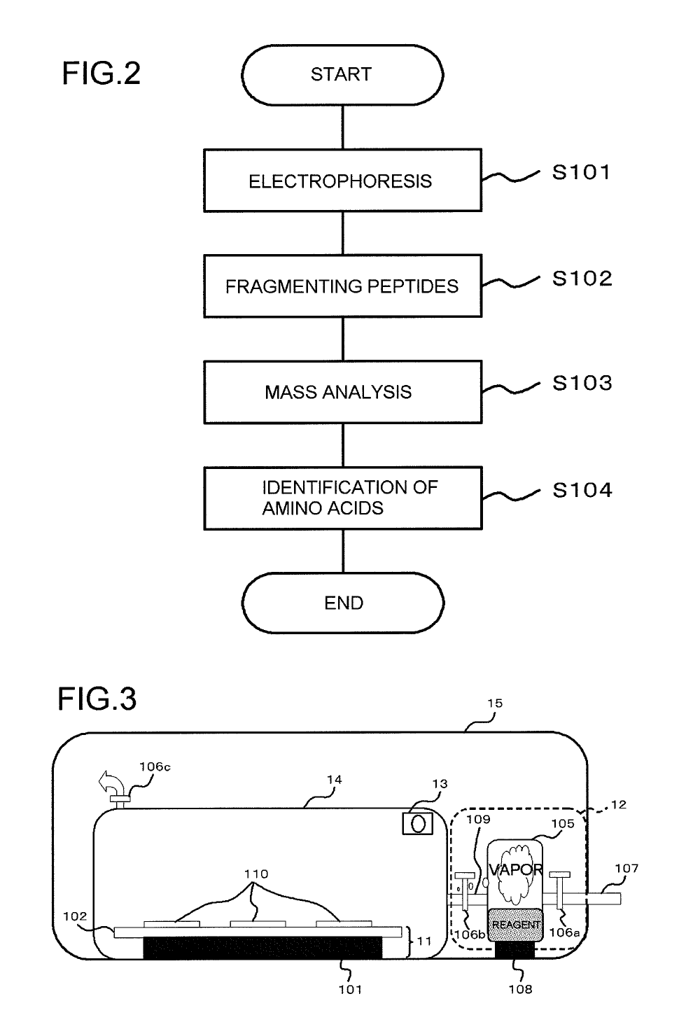

Here, the method for degrading peptides of the present exemplary embodiment may be employed in the method for analyzing peptides, procedure of which is shown in FIG. 2. Such analyzing method involves supplying a sample serving as an object of the measurement (protein or peptide) in flow paths to cause electrophoresis (S101). Subsequently, fragmentation reaction of peptide fractions isolated via electrophoresis is conducted (S102). Then, mass analysis of fragmented peptide fractions is conducted (S103). Then, data matching with the sequence information in the database is conducted to identify amino acid sequence (S104).

Method for degrading peptides of the present exemplary embodiment is applicable to an operation for causing electrophoresis (S101) and fragmentation reaction for peptides (S102) shown in FIG. 2. First of all, an operation for causing electrophoresis (S101) will be described.

A sample solution containing proteins or peptides is introduced into an appropriate flow path. Subsequently, isoelectric focusing is conducted to isolate the sample into two or more peptide fractions to give isolated peptide fractions in flow paths (S110).

Then, a drying process is conducted while maintaining respective peptide fractions in the flow path to volatilize a solvent (S111). The drying process is preferably conducted by a freeze-dry process. In this case, a buffer may be added in the solvent. Preferable quantity of the buffer may be a level, which does not affect the mass analysis. Typical buffer includes ammonium hydrogen carbonate and tris hydrochloric acid buffer. This allows inhibiting inactivation of protease by autolysis thereof when respective peptide fractions are in contact with protease, as will be discussed later.

Next, fragmentation reaction for peptides (S102) will be performed. Obtained dried peptides are brought into contact with protease (S112). Typical protease available here may include, for example, trypsin, lysyl endopeptidase and V8 protease. Protease is an enzyme, which accelerates hydrolysis of peptide bond in specified amino acid site in peptide. Therefore, fragmentation of peptides is caused by acting protease on peptide in the presence of water. Therefore, a presence of water is an essential factor for fragmentation reaction of peptides with protease, and no fragmentation reaction is caused even if protease is activated with peptide under an absence of water. Therefore, when dried peptide is brought into contact with protease, it is preferable to conduct the contacting process in an environment containing less water as possible. If a presence of water is unavoidably required, it is preferable to carry out the operation in an environment of a temperature, which is not an optimal temperature for the protease.

The following forms are expected as approaches for contacting peptide fractions with the protease:

(1) an approach for applying a protease solution over a surface of dried peptide; and

(2) an approach for mixing a powdered protease with dried peptide.

Each of these forms will be sequentially described as follows.

### [(1) Approach for Applying Protease Solution Over a Surface of Dried Peptide]

First of all, a protease solution containing a protease dissolved therein is prepared. Then, the protease solution is applied over each of the peptide fractions. Subsequently, the peptide fractions applied with the protease solution are dried.

In such case, it is necessary to promptly volatilize solvent so as to prevent from mixing respective peptide fractions in the case that the protease solution is applied. Therefore, it is preferable to dissolve the protease in a volatile organic solvent to prepare the protease solution. Typical volatile organic solvents available here include, for example, acetonitrile, methanol and the like. When the dissolution of the protease is not sufficient only with the volatile organic solvent, water may be optionally added.

In addition, the protease solution may contain an inorganic salt. Typical inorganic salts available here include, for example, ammonium hydrogen carbonate. This allows achieving appropriate pH for the protease in the reaction field in case of the fragmentation reaction as discussed later. Deactivation by autolysis can be reduced by promptly volatilizing the solvent after applying the protease solution.

[(2) Approach for Mixing Dried Peptide with the Protease Powder]

First of all, the protease is freeze-dried to prepare the protease powder. Subsequently, each of peptide fractions is mixed with the protease powder.

In such case, the protease powder may be prepared by dissolving the protease in the volatile organic solvent, and cooling the solution to an ultracold temperature with liquid nitrogen or the like, and then crushing them with a mortar to prepare a frozen powder. The protease powder thus prepared is supplied into the flow paths, and then is heated to a temperature within a range of from a room temperature (25 degrees C.) to 100 degrees C. This allows substantially simultaneously achieving the dissolution of the protease powder and the evaporation of the solvent. This results in the preparation of a protease/peptide mixture-dried material.

In order to avoid mixing each of the peptide fractions, the solvent contained in the protease powder is required to be immediately volatilized. Therefore, the protease is preferably dissolved in a volatile organic solvent. Typical volatile organic solvents available here include, for example, acetonitrile, methanol and the like. When the dissolution of the protease is not sufficient only with the volatile organic solvent, water may be optionally added.

When the protease powder is prepared, suitable inorganic salt may be contained. Typical inorganic salts include ammonium hydrogen carbonate. This allows achieving appropriate pH for the protease in the reaction field in case of the fragmentation reaction as discussed later.

As described above, the dried peptide contacted with the protease may be set in a device for degrading peptide 1 shown in FIG. 3 by each of the flow paths.

The device for degrading peptide shown in FIG. 3 includes a sample holding unit 11 for holding flow paths containing dried mixed sample, which contains a plurality of peptide fractions isolated via electrophoresis and a protease that is in contact with peptide fractions, a vapor supplying unit 12 for supplying water vapor in the flow paths, and a sensor 13 for detecting formations of respectively independent liquid membranes of a solvent by the supplied water vapor over the surfaces of the peptide fractions that have been in contact with the protease for each of flow paths.

The device for degrading peptide 1 includes the vapor supplying unit 12 and a chamber 14 (reaction vessel) in an incubator 15. The chamber 14 includes the sample holding unit 11.

The temperature in the incubator 15 may be suitably controlled to control a temperature of a reagent supplied as a vapor from a reagent vessel 105, so that an evaporation or a volatilization of the reagent over the peptide fractions can be appropriately achieved.

The sample holding unit 11 may include a Peltier element 101. This allows heating and cooling the flow paths. Therefore, the temperature of the flow path can be controlled, such that the reagent is supplied in a form of vapor from the reagent vessel 105 may be deposited over the peptide fractions or volatilized. The flow paths may be preferable heated to an optimal temperature for the protease. This allows initiating the reaction promptly after the liquid membrane is formed. In addition, the sample holding unit 11 has an anticorrosive block 102. This prevents from a formation of a nonvolatile matter by corrosion and a contamination to the sample. The block 102 may support chips 110 for electrophoresis that include flow paths as shown in FIG. 3.

The vapor supplying unit 12 includes a reagent vessel 105, solenoid valves 106a and 106b, an inert gas pipe 107, a Peltier element 108, and a reagent vapor supplying line 109. The solenoid valve 106a provides a control of a flow rate of the inert gas. This allows depositing the reagent supplied from the reagent vessel 105 in a form of a vapor over the peptide fractions, or volatilizing thereof from the peptide fraction. In addition, the solenoid valve 106b may provide a control of a supply level of the inert gas in addition to the supply level of water vapor.

Water is supplied in the reagent vessel 105, and then the Peltier element 108 is energized to heat the reagent vessel 105.

This allows the vapor supplying unit 12 supplying water vapor in the chamber 14.

A basic or an acid volatile organic solvent may be added to the reagent vessel 105. In such case, it is particularly preferable to employ a basic nitrogen-containing aromatic compound.

Typical basic nitrogen-containing compound available here may include heterocyclic compounds such as pyridine, collidine and the like. This allows suitably controlling pH condition in the fragmentation reaction to an optimum pH for the protease. In addition, a need for conducting a desalting process is omitted, so that better signal/noise (S/N) ratio can be achieved when a mass analysis is conducted. Typical basic or acid volatile organic solvents include, for example, pyridine/acetic acid buffer, pyridine/collidine/acetic acid buffer and the like. Such buffer may be supplied with water vapor to form liquid membrane containing an acid or a basic volatile organic solvent over peptide fractions. This allows locating each of peptide fractions in a preferable pH condition, effectively promoting the fragmentation reaction, while avoiding diffusion/contamination of peptide fractions.

In addition, an inert gas may be supplied therein from an inert gas pipe 107. This allows supplying an inert gas into the chamber 14 from the vapor supplying unit 12.

In addition, the solenoid valves 106a and 106b may be suitably adjusted to supply an inert gas with water vapor into the chamber 14 from the vapor supplying unit 12. This allows supplying water vapor with a carrier gas of an inert gas in the flow paths. The liquid membrane is formed to contain sufficient levels of water molecules for promoting the reaction. In addition, the thickness of the liquid membrane is controlled as long as no diffusion of the respective peptide fractions or no contaminations among the samples is caused. A quantity of water vapor contained in the carrier gas may be controlled so that the thickness of the membrane can be easily controlled.

Typical inert gases may include nitrogen gas and argon gas.

A charge-coupled device (CCD) camera, for example, may be employed for the sensor 13. In this case, it is preferable to store the optimum thickness of the liquid membrane, and to detect the condition, in which suitable size of the liquid membrane is attained. In addition, the sensor 13 may be a sensor that can detect the vapor concentration. In this case, quantity of vapor required for forming suitable size of liquid membrane is previously acquired via experiments or calculations. Then, the sensor 13 detects the condition, in which suitable vapor quantity is attained in the chamber 14. The configuration of the sensor 13 may also be set to stop the supply of argon gas by energizing the solenoid valve 106a to provide a shut state for the interior of the chamber 14, when the sensor detects that the suitable size of the liquid membrane is attained or suitable quantity of vapor is attained in the chamber 14. In such case, the temperature of the sample holding unit 11 may be automatically increased.

The fragmentation reaction for the dried peptide set in the device for degrading peptide 1 may be progressed by the following routine.

First of all, water vapor is generated from the vapor supplying unit 12 to supply water vapor into the chamber 14. In this occasion, the vapor supplying unit 12 is capable of supplying water vapor with an inert gas. This allows supplying proper quantity of water vapor into the chamber 14 to form liquid membrane over the surface of each of the peptide fractions. When an excess quantity of water vapor is supplied, the formed liquid membrane may extend to overlap two or more peptide fractions. This is not preferable since a contamination among peptide fractions is occurred.

On the other hand, insufficient thickness of the formed liquid membrane is not preferable. The reason is that the fragmentation reaction of peptides proceeds by hydrolysis reaction.

Therefore, a formation of the liquid membrane containing a quantity of water molecules required for the reaction is suitable in principle.

Quantity of supplied water vapor may be determined according to, for example, the size of a vapor droplet deposited on the surface of peptide fractions. More specifically, the quantity may be determined so that the particle size of the vapor droplets expanding by fusion of the vapor droplets does not disturb the isolated state of the samples, or so that the spatial concentration of the samples is not reduced to a level less than the measurement limit.

Subsequently, when the sensor 13 detects that respectively independent liquid membranes are formed on the surface of each of the peptide fractions(S113), the chamber 14 is tightly closed to maintain the formation of the liquid membranes (S114). More specifically, the sensor 13 may control the solenoid valve 106a when the grow of the droplets to a certain dimension is detected after the adhension of the droplets thereto to increase a flow rate of inert gas or the like, reducing the quantity of water vapor included in the inert gas. This allows filling the chamber 14 with the inert gas and water vapor. Further, the temperature in the chamber 14 may be selected so as to avoid evaporating the formed liquid membrane. For example, the temperature of the Peltier element 101 of the sample holding unit 11 may be selected to be slightly higher than the optimal temperature. This allows increasing the temperature of the liquid membrane, so that the diameter of the vapor droplet is not expanded or shrunk without controlling the quantity of vapor with the carrier gas. Then, the solenoid valve 106b is shut to tightly close the flow path in the chamber 14, thereby maintaining the formation of the liquid membrane. In this way, the dimension of the formed liquid membrane can be maintained, the fragmentation reaction can be progressed while preventing a diffusion and a contamination of peptide fractions.

Then, the formation of the liquid membrane of the solvent is maintained for a predetermined time, so that protease reacts on peptides in the respective peptide fractions, and thus the fragmentation reaction is progressed. The fragmentation reaction is practically achieved in approximately 10 minutes. However, the retention time may be extended to an arbitrary duration time, as long as the quantity of water is adequately controlled.

As such, after the progress of the arbitrary retention time, the solenoid valve 106c is opened to open the chamber 14, and the solvent is vaporized while supplying inert gas to remove the liquid membrane (S115). The solenoid valve 106c is opened to exhaust the gas in the chamber 14 to the exhaust line. This causes regaining the dryness state, thereby stopping the fragmentation reaction. In such case, the electric current through the Peltier element 101 may also be suitably controlled to increase the temperature of the sample holding unit 11 to above a certain level. This allows an inactivation of the protease. Further, when remained salt is a volatile material or is susceptible to a thermal decomposition, the temperature may be maintained to an elevated temperature higher than the decomposition temperature for an arbitrary duration time to achieve a desalination.

Subsequently, a matrix is mixed with the fragmented peptide fraction to conduct a mass analysis (S103). Then, data matching with the sequence information in the database is conducted to identify amino acid sequence (S104).

In such case, an aqueous solution containing an organic solvent, which is prepared by mixing the matrix therewith, may be applied over the dried material of the fragmented peptide fraction. Subsequently, the organic solvent water solution is promptly dried to prepare a crystalline mixture of the digested fragment peptide derived from the peptide and the matrix.

Alternatively, for the dried material of the fragmented peptide fraction, a matrix powder may be put on the dry matter of the fragmented peptide fraction. The matrix powder may be prepared by dissolving the matrix in a water solution containing an organic solvent and frozen at a cryogenic temperature utilizing liquid nitrogen or the like to solidify thereof, and further crushing the frozen material utilizing a mortar or the like to obtain frozen powder. The matrix powder is put uniformly over the surface of the dried matter of the fragmented peptide fraction, and then is promptly heated to a temperature within a range of from a room temperature (25 degrees C.) to 100 degrees C., so that a fusion with the fragmented peptide fraction and a prompt volatilization of the organic solvent-aqueous solution are caused. As such, the crystalline mixture of the fragmented peptide fraction and the matrix may be prepared.

In addition, the device for degrading peptide 1 may be connected to a mass spectrometer for conducting a mass analysis of the peptide fraction to utilize as a device for analyzing peptide.

In addition to above, the method of the present exemplary embodiment may be applicable to other cases except for the peptide fractions isolated via an electrophoresis. In addition, the fragmentation reaction of the present exemplary embodiment may be carried out in other atmospheres except the inside of the flow paths for the electrophoresis. For example, two or more peptides, which are previously isolated by a certain method may be applicable to the peptide fractions prepared in a sample plate of matrix assisted laser desorption ionization (MALDI)-time of flight (TOF)-mass spectrometry (MS). This allows fragmenting peptides disposed on the plate as a sample for the mass analysis without causing a loss.

Next, advantageous effects obtainable by employing the method for degrading peptides of the present exemplary embodiment will be described. According to the present method, two or more peptide fractions isolated via an electrophoresis are dried for each of the flow paths, and then are brought into contact with the protease. Then, respective independent liquid membranes of a solvent are formed on the flow paths over the surface of the peptide fractions. This allows the liquid membranes formed on the surface of respective the peptide fractions functioning as a solvent, causing the protease acting on the peptides. Therefore, the fragmentation reaction can be progressed independently by each of the peptide fractions, and a fragmenting reaction of peptide can be achieved effectively in a short time while maintaining the isolated state of peptide.

In addition, according to such method, liquid membranes of a solvent can be formed over the surfaces of the respective peptide fractions isolated via an electrophoresis. This allows reducing a required quantity of the solvent, thereby reducing a diffusion and/or a loss of the peptide fractions to a practical level. In addition, wider dimensional areas for contact surfaces among peptides, protease and water can be utilized, achieving an effective progress of the reaction and a decrease of the time required for the reaction. Then, such fragmentation with higher efficiency promotes improved sensibility and accuracy of a mass analysis, so that an efficiency in an identification operation for a sample of peptide or protein can be entirely achieved.

Conventionally, when a fragmentation of peptides is conducted by employing protease, complicated sorting operation is required for conducting the fragmentation if the fragmentation is carried out while maintaining the isolated state of peptides. Therefore, labor and time are required for the fragmentation reaction of peptides. In addition, such operations may cause losses and contaminations of the samples, and thus causing a number of problems. Therefore, an establishment of a process for fragmenting peptides without causing a loss and/or a mixing of a sample and with less labor operation is expected.

On the contrary, according to the method of the present exemplary embodiment, protease and peptide are previously mixed under a dried status during the fragmentation process. Then, the liquid membrane of a solvent can be formed over such protease-peptide mixture in the dried states. This allows reducing a fluidization and/or a diffusion of the sample.

The liquid membrane may be, in principle, formed by a manual operation over the surface of the fractions of the respective peptides by employing, for example, a micropipette or the like. Nevertheless, the formation of the liquid membrane by utilizing water vapor allows suitably heating the respective fractions while supplying a suitable quantity of water for the peptide fraction. Therefore, the fragmentation reaction can be achieved at a temperature within the range of the optimal temperature for the protease. Therefore, the fragmentation reaction can be more rapidly progressed with higher efficiency.

Further, the configuration of the present exemplary embodiment may be preferably employed for a gel-free electrophoresis. In the gel-free electrophoresis, in order to prevent a diffusion in the electrophoresis, a solution having a certain level of viscosity is employed to conduct a freeze-dry process promptly after the electrophoresis. When a fragmentation with enzyme is carried out for the protein isolated by such gel-free electrophoresis, a diffusion of the isolated peptide fractions is easily occurred. However, in the present exemplary embodiment, the dried peptide fractions are brought into contact with protease, and then the independent liquid membranes of a solvent are formed over the surfaces of peptide fractions, respectively. This allows the liquid membranes formed on the surface of the respective peptide fractions functioning as a solvent, causing the protease acting on the peptides. Therefore, the fragmentation reaction can be progressed independently by each of the peptide fractions, and a fragmenting reaction of peptide can be achieved effectively in a short time while maintaining the isolated state of peptide.

More specifically, in an effective enzymatic hydrolysis process for peptides of the present exemplary embodiment, water, which is considered as being essential for the fragmentation reaction of the protease for peptides, is removed from the reaction systems in the initial stage of the reaction, and then water is supplied in a form of water vapor in the reaction system while maintaining the optimum reaction temperature. The quantity of the supply of the water is suitably controlled by controllably supplying a necessary quantity of water for promoting the reaction, and is controlled so as to avoid an increase of flowability due to retention of water in the reaction system beyond necessity. This allows preventing from a diffusion of the peptide fractions or a contamination of the adjacent peptide fractions. Therefore, the mass analysis with higher sensitivity can be achieved.

As described above, according to the method of the present exemplary embodiment, even if the peptide fractions are isolated and located adjacent on the chip for isolating protein, for example, it is possible to act the protease over the peptide fractions while maintaining the isolated state of the peptide fractions. In addition, the reaction may proceed in short duration time as compared with an ordinary fragmentation reaction. In addition, since the fragmentation reaction is achieved in the flow paths, the sample can be directly measured in the mass analyzer without any modification, allowing database search for proteins with higher efficiency. Further, since the isolated state is maintained, a protein analysis can be achieved with higher sensibility and/or accuracy.

While the exemplary embodiments according to the present invention have been described in reference to the annexed drawings, it is understood that these are the illustrations of the present invention, and other configurations except the above-described exemplary embodiments are also available.

For example, the present invention may alternatively be applied to the following configurations.

(1) A technique for hydrolyzing of an analysis target of peptides employing protease, wherein an appropriate temperature for proceeding the reaction for peptide and protease coexisting in a dried state in at least the reaction system can be maintained, and vapor formed from an arbitrary solvent is supplied to the reaction system directly or with an inert gas to achieve a regulation and/or a control of an absolute quantity of the solvent carried in the reaction system, and wherein the effect of such regulation and/or control allows a hydrolysis of the peptide with less diffusion of reaction products and less contamination among samples.

(2) The method for hydrolyzing peptide as set forth in (1), wherein the solvent for promoting the hydrolysis of peptide contains aqueous solution containing volatile organic solvent.

(3) The method for hydrolyzing peptide as set forth in (2), wherein the volatile organic solvent for promoting the hydrolysis of peptide contains a basic nitrogen-containing aromatic compound.

(4) The method for hydrolyzing peptide as set forth in any of (1) to (3), wherein, on the occasion of the regulation and/or the control of the above-described quantity of the solvent conducted for reducing the diffusion of the reaction product, a difference in the temperature between the solvent and the reaction system is utilized.

(5) The method for hydrolyzing peptide as set forth in any of (1) to (4), wherein, on the occasion of the regulation and/or the control of the above-described quantity of the solvent conducted for reducing the diffusion of the reaction product, a quantity of an inert gas carrying a solvent vapor into the reaction system is increased or decreased to control a concentration of the solvent vapor existing in the inert gas, thereby adequately controlling the quantity of the solvent retained in and acting over, the reaction system.

(6) The method for hydrolyzing peptide as set forth in any of (1) to (4), wherein, on the occasion of the regulation and/or the control of the above-described quantity of the solvent conducted for reducing the diffusion of the reaction product, a temperature of the solvent for supplying the solvent vapor is increased or decreased to control the concentration of the solvent vapor existing in the inert gas, thereby adequately controlling the quantity of the solvent acting over the reaction system.

(7) The method for hydrolyzing peptide as set forth in any of (1) to (6), wherein, trypsin is employed for the protease.

(8) The method for hydrolyzing peptide as set forth in any of (1) to (6), wherein, a chip for isoelectric focusing for isolating protein is employed in the reaction system.

(9) The method for hydrolyzing peptide as set forth in (8), wherein the method for supplying protease includes: applying, with a solvent, a protease, which has been diluted in an buffer at an appropriate concentration, and then: promptly volatilizing the solvent to prepare a dried mixture of the peptide and the protease and at the same time, to remove the solvent from the reaction system.

(10) The method for hydrolyzing peptide as set forth in (8), wherein the method for supplying protease includes:

cooling a protease, which has been diluted in an buffer at an appropriate concentration, to obtain a solid material; crushing the solid material into powder under an environment for preventing a deposition of water, in which water is removed within an atmosphere of an inert gas; mixing the powder with dried peptide that has been cooled in the same temperature therewith within an atmosphere of an inert gas;

rapidly elevating the temperature in shorter time to liquidize the buffer and simultaneously bringing protease and peptide in a solution state; and then promptly vaporizing the solvent to prepare a dried blended material of protease and peptide.

Further, the present invention may alternatively be applied to the following configurations.

(11) A method for degrading peptide utilizing protease, including a degradation process, in which vapor created from an arbitrary solvent is supplied over the peptide and the protease coexisting in a dried state to cause the protease degrading the peptide, wherein quantity of the vapor employed in the degradation process is controlled.

(12) The method for degrading peptide as set forth in (11), wherein the quantity of the vapor is determined according to the size of a vapor droplet deposited over protease and peptide, and the size of the vapor droplet is a suitable size for controlling a diffusion of the protease and the peptide.

(13) The method for degrading peptide as set forth in (11) or (12), wherein the vapor is supplied with an inert gas.

(14) The method for degrading peptide as set forth in any of (11) to (13), wherein the solvent is an aqueous solution containing a volatile organic solvent.

(15) The method for degrading peptide as set forth in (14), wherein the volatile organic solvent contains a basic nitrogen-containing aromatic compound.

(16) The method for degrading peptide as set forth in any of (11) to (15), wherein the quantity of the vapor is determined by utilizing a difference between the temperature of peptide and protease and the temperature of the solvent.

(17) The method for degrading peptide as set forth in any of (11) to (16), wherein the quantity of the vapor supplied in the process for supplying the vapor is controlled by controlling the quantity of the inert gas.

(18) The method for degrading peptide as set forth in any of (11) to (17), wherein the quantity of the vapor employed in the degradation process is controlled by controlling the temperature of the vapor.

(19) The method for degrading peptide as set forth in any of (11) to (18), wherein the degradation process is carried out in a flow path of a chip for isoelectric focusing.

(20) The method for degrading peptide as set forth in any of (11) to (19), wherein the peptide is migrated in the flow path of the chip for isoelectric focusing.

(21) The method for degrading peptide as set forth in any of (11) to (20), further including: a dissolution process for dissolving the protease in the buffer solution;

a mixing process for mixing the peptide with the buffer solution; and

a volatilization process for volatilizing the buffer solution containing the protease and the peptide to form the peptide and the protease coexisting in a dried state.

(22) The method for degrading peptide as set forth in any of (11) to (21), further including:

a dissolution process for dissolving the protease in the buffer solution;

a cooling process for cooling the buffer solution to form solidified protease;

a mixing process for mixing the solidified protease and the peptide;

a dissolution process for dissolving the solidified protease in the buffer solution to mix the protease and the peptide in the buffer solution; and

a volatilization process for volatilizing the buffer solution containing the protease and the peptide to form the peptide and the protease coexisting in a dried state.

(23) A method for analyzing peptide, wherein peptide degraded by the method for degrading peptide as set forth in any of (11) to (22) is analyzed by employing a mass analyzer.

(24) A device for degrading peptide utilizing protease, including:

a sample holding unit for holding peptide and protease coexisting in a dried state; and

a vapor supplying unit for supplying vapor created from an arbitrary solvent over the peptide and the protease,

wherein the vapor supplying unit controls the quantity of the vapor.

In addition to above, it is needless to point out that the above-described exemplary embodiment and a plurality of modified exemplary embodiments may be combined unless the configurations are not contradicting.

In addition, while the structures of the respective sections have been specifically described in the above-described exemplary embodiment and modified exemplary embodiments the structures may alternatively be modified within a range for satisfying the present invention.

### EXAMPLES

Examples will be presented as follows for further describing the present invention. It is intended that the scope of the present invention is not the limited to such examples.

### Example 1

For the purpose of studying an effectiveness of the present invention, commercially available equine apomyoglobin or bovine carbonic anhydrase was introduced into flow paths by employing a chip for isoelectric focusing. Subsequently, a freeze-dry process was conducted to prepare dried protein. A trypsin solution (water: acetonitrile=3:7, ammonium hydrogen carbonate 0.5 mM) was sprayed over the thus obtained dried protein in the flow path, and the chip for the isoelectric focusing was previously heated to 80 degrees C. This operation allowed rapidly drying the solvent, preparing a protein/trypsin dried mixture in the flow path. A proper amount of water vapor was supplied over the obtained protein/trypsin dried mixture to avoid a diffusion of the reaction product while promoting the enzyme reaction.

In the present example, equine apomyoglobin and bovine carbonic anhydrase were employed for the analysis targets. Then, the protein was introduced in the flow path utilizing the chip for isoelectric focusing and was dried, and then, trypsin, which was dissolved in an organic solvent, was applied, and then was rapidly dried to prepare an enzyme/protein dried mixture. Subsequently, peptide fragment was generated by the method of the present exemplary embodiment, and the molecular weight of the generated peptide fragment was measured. The present example will be described as follows.

(Preparation of Dried Protein with Chip for Isoelectric Focusing)

For commercially available equine apomyoglobin sample, a peptide aqueous solution containing only apomyoglobin chain site was prepared at a concentration of 3 μg/μL. The peptide aqueous solution was supplied into a flow path of the chip for the protein isoelectric focusing, and then was frozen at a temperature of −25 degrees C. to carry out a freeze drying to obtain the dried peptide. Further, for commercially available bovine carbonic anhydrase sample, a peptide aqueous solution containing only carbonic anhydrase chain site was prepared at a concentration of 4 μg/μL. Such peptide aqueous solution was supplied into a flow path of the chip for the protein isoelectric focusing, and then was frozen at a temperature of −25 degrees C. to carry out a freeze drying to obtain the dried peptide.

### (Preparation of Trypsin-Acetonitrile Aqueous Solution)

On the contrary, for trypsin, an acetonitrile aqueous solution (acetonitrile: water=7:3) of trypsin at a concentration of 60 ng/μL was prepared at an ice temperature. In such acetonitrile aqueous solution, ammonium hydrogen carbonate was previously added to be a concentration of 0.5 mM.

### (Preparation of Dried Mixtures of Trypsin and Apomyoglobin and Trypsin and Carbonic Anhydrase)

The above-described trypsin solution was applied over the above-described dried peptide at an amount of about 10 μL per each of flow paths. In such case, the temperature of the chip for the protein isoelectric focusing was set to 80 degrees C., so that the acetonitrile solution of trypsin was promptly dried.

### (Supply of Water Vapor and Enzyme Reaction)

Water vapor was supplied for the above-described trypsin-peptide dried mixture by employing a device shown in FIG. 3.

Argon was used as a carrier gas, and a flow rate thereof was about 0.5 L/min. The chip for the protein isoelectric focusing was supported in the sample holding unit 11, and the temperature of the sample holding unit 11 was set to 37 degrees C., and the unit was disposed in the chamber 14 having a volume of about 2 L, and then water vapor was supplied from the vapor supplying unit 12. More specifically, water was supplied to a reagent vessel 105 of the vapor supplying unit 12, and the temperature of the reagent vessel 105 was set to 57 degrees C. A formation of the liquid membrane of the solvent formed independently by the peptide fractions was detected, and after that, the temperature in the device 1 for degrading peptide was maintained at 60 degrees C. during the entire process. The actual value of the temperature was 58 degrees C. The supply of argon gas was stopped at the time when the surface of the chip for the protein isoelectric focusing was started to be misted by the condensed water vapor, and then the chamber 14 was closed, and the closed state was maintained for 16 hours. Thereafter, the supply of argon gas was started again, and the temperature of the reagent vessel 105 was set to 15 degrees C. and the supply of water vapor was stopped, so that the chip for protein isoelectric point electrophoresis was changed to the dried state again.

### (Application of Matrix)

An acetonitrile aqueous solution of sinapinic acid (acetonitrile: water=7:3, containing 0.05% of trifluoroacetic acid (TFA)) was prepared, and the prepared solution was applied over the chip for the protein isoelectric focusing, which was in a condition that the above-described enzyme reaction was completed. The temperature of the chip for the protein isoelectric focusing was maintained at 80 degrees C. during the applying operation to rapidly volatilize the solvent.

### (Mass Analysis)

The mass of peptide fragments was measured by employing a matrix-assisted laser desorption/ionization time of flight mass spectrometer (MALDI-TOFMS) for the chip for the protein isoelectric focusing after the application of the above-described sinapinic acid.

### (Results)

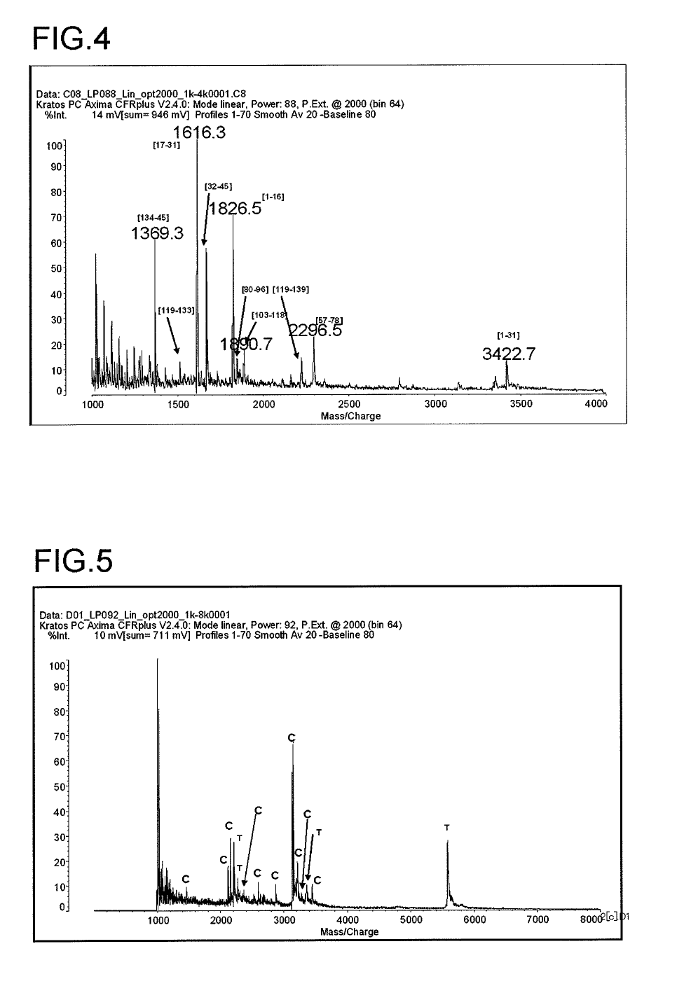

A spectrum of fragmented peptide by trypsin obtained from equine apomyoglobin is shown in FIG. 4. A spectrum of fragmented peptide by trypsin obtained from bovine carbonic anhydrase is shown in FIG. 5. In FIG. 5, “C” indicates a peak derived from carbonic anhydrase, and “T” indicates a peak derived from trypsin.

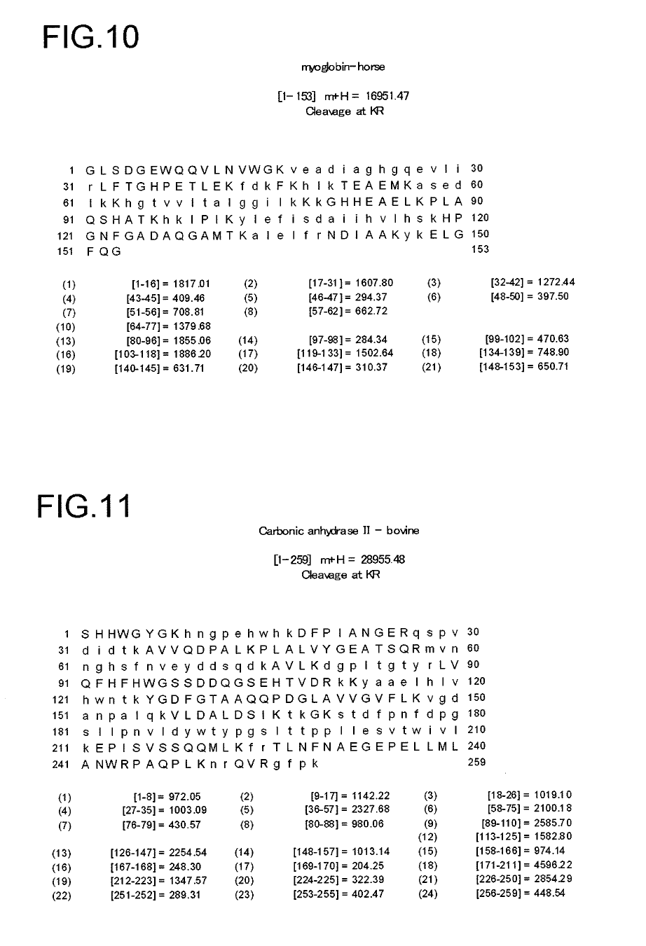

FIG. 10 shows amino acid sequence of equine apomyoglobin and fragment obtained by breaking equine apomyoglobin with trypsin, and molecular weights thereof (MH⁺). FIG. 11 shows amino acid sequence of bovine carbonic anhydrase and fragment obtained by breaking bovine carbonic anhydrase with trypsin, and molecular weights thereof (MH⁺). It is understood by the comparison of FIG. 4 and FIG. 10 that a molecular weight which coincides with the molecular weight of peptide fragment shown in FIG. 10 is actually measured in the spectrum of FIG. 4. It is also understood by the comparison of FIG. 5 and FIG. 11 that a molecular weight which coincides with the molecular weight of peptide fragment shown in FIG. 11 is actually measured in the spectrum of FIG. 5. Therefore, it is shown that the peptide derived from the digested fragment peptide are observed in both of FIG. 4 and FIG. 5, and thus it is shown that the technique of the present invention is effectively workable.

### Example 2

For bovine carbonic anhydrase, approximately 5% in the gross quantity of bovine carbonic anhydrase was dyed with commercially available Cy3 fluorochrome (commercially available from GE Healthcare), and then was introduced into a flow path of a chip for the protein isoelectric focusing. A voltage was applied to converge into an isoelectric point, and then a freeze-dry process was conducted to prepare dried protein in one point in the flow path. Here, the Cy3 fluorochrome employed in such case was the product that was guaranteed by the manufacturer as presenting no influence in the isoelectric point of protein. In other words, while the rate of the protein on the flow path detected via fluorescence is 5% of the whole protein, it was ensured that the remaining 95% of protein, which were not labeled with the fluorescent pigment, was also converged in the isoelectric point site at which the fluorescence was detected.

(Fluorescent Labeling of Bovine Carbonic Anhydrase with Cy3)

A fluorescent labeling and a desalination of a commercially available bovine carbonic anhydrase were carried out with a commercially available Cy3 to present a concentration of 1 μg/μL.

(Isoelectric Focusing of Cy3-Labeled Carbonic Anhydrase with Chip for Isoelectric Focusing)

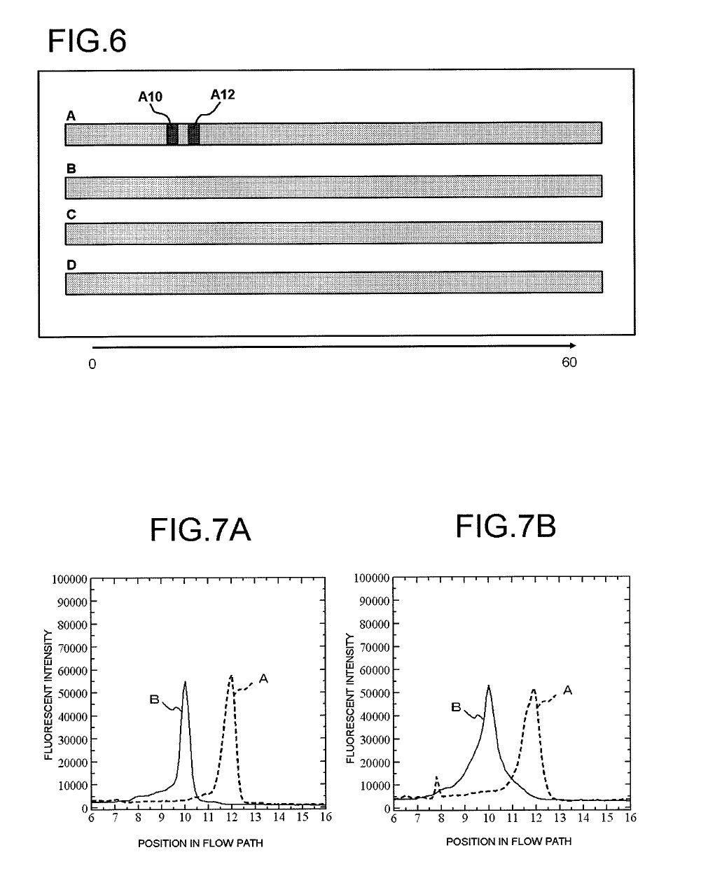

2 μL of the above-described Cy3-labeled carbonic anhydrase, 47 μL of capillary isoelectric focusing (cIEF) gel (commercially available from Beckman Coulter), and 1 μL of carrier ampholyte (pI3-10) were mixed, and then 0.25 μL of the mixture was introduced into flow paths of a chip for isoelectric focusing to carry out an isoelectric focusing. The chip for isoelectric focusing employed in such case is shown in FIG. 6. As illustrated here, the chip has flow paths corresponding to four channels of A, B, C and D, and was configured so that an electrophoresis of each sample can be achieved in each of the flow paths. In order to designate locations of the electrophoresis in each of the channels, the length of the channel was equally divided by 61, and each of the divided segments are assigned to numbers of 0, 1, 2 to 60 from the left side. The both end sections of 2.5 mm from each of the ends of one channel are not assigned, because these sections are out of the measurement. In FIG. 6, only A10 and A12 in the flow path “A” are shown.

An isoelectric focusing was carried out, and then a freeze-dry process was conducted for the whole chip. FIG. 7A shows a result of a scanning along the flow path with a fluorescence microscope after the freeze-dry process. FIGS. 7A and 7B show both of the results for the A flow path and the B flow path.

It was found that carbonic anhydrase converged near a flow path position 12 in the A flow path, and near a flow path position 10 in the B flow path.

### (Preparation of Trypsin-Acetonitrile Aqueous Solution)

For trypsin, an acetonitrile aqueous solution (acetonitrile: water=7:3) of trypsin at a concentration of 60 ng/μL was prepared at an ice temperature. In such acetonitrile aqueous solution, ammonium hydrogen carbonate was previously added to be a concentration of 0.5 mM.

### (Preparation of Dried Mixtures of Trypsin and Carbonic Anhydrase)

The above-described trypsin solution was applied over the above-described dried peptide at an amount of 10 μL per each of flow paths. In such case, the temperature of the chip for the protein isoelectric focusing was set to 80 degrees C., so that the acetonitrile solution of trypsin was promptly volatilized and dried.

### (Supply of Water Vapor and Enzyme Reaction)

Water vapor was supplied for the above-described trypsin-peptide dried mixture by employing a device as shown in FIG. 3. The temperature of the incubator 15 was set to 60 degrees C. The actual value of the temperature was 58 degrees C. Argon was used as a carrier gas, and a flow rate thereof was about 0.5 L/min. The chip for the protein isoelectric focusing was supported in the sample holding unit 11, and the temperature of the sample holding unit 11 was set to 37 degrees C., and the unit was disposed in the chamber 14 having a volume of about 2 L, and then water vapor was supplied from the vapor supplying unit 12. More specifically, water was supplied to a reagent vessel 105, and the temperature of the reagent vessel was set to 57 degrees C. A formation of the liquid membrane of the solvent formed independently by the peptide fractions was detected, and after that, when the surface of the chip for the protein isoelectric focusing was started to be misted by the condensed water vapor, the temperature of the reagent vessel was decreased to 15 degrees C, and the supply of water vapor was stopped, and the chip for protein isoelectric point electrophoresis was changed to the dried state again. The trypsin-peptide dried mixture was maintained under a wet condition by the supplied water vapor at a temperature of 37 degrees C for substantially 15 minutes. The chip for the protein isoelectric focusing, which was in the dried condition again, was taken out, and the scanning was conducted along the flow path with a fluorescence microscope. The result is shown in FIG. 7B. While the convergence condition was slightly moderated due to the application of trypsin acetonitrile solution and the supply of water vapor, the position of the center is not deviated.

### (Application of Matrix)

An acetonitrile aqueous solution of sinapinic acid (acetonitrile: water=7:3, containing 0.05% of TFA) was prepared, and the prepared solution was applied over the chip for the protein isoelectric focusing, which was in a condition that the above-described enzyme reaction was completed. The temperature of the chip for the protein isoelectric focusing was maintained at 80 degrees C. during the applying operation to rapidly volatilize the solvent.

### (Mass Analysis)

The mass of reaction products was measured by employing a matrix-assisted laser desorption/ionization time of flight mass spectrometer (MALDI-TOFMS) for the chip for the protein isoelectric focusing after the application of the above-described sinapinic acid.

### (Results)

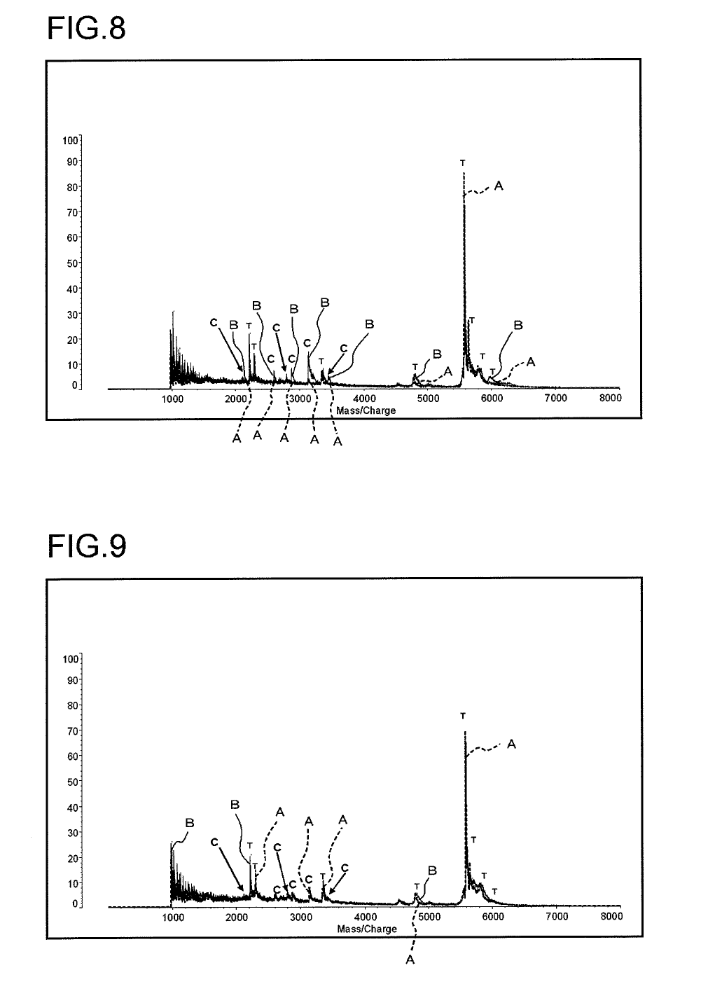

A spectrum of the mass analysis obtained from peptide fraction of near the flow path position A10 is shown in FIG. 8. In FIG. 8, “C” indicates a peak derived from carbonic anhydrase, and “T” indicates a peak derived from trypsin. FIG. 8 shows both of the results for the A flow path and the B flow path. While it is known from the results of FIG. 7 that carbonic anhydrase is converged in the flow path B near the position, it is understood that the signal of peptide fragment obtained by a tryptic digestion of carbonic anhydrase is also strongly observed from the flow path B. A spectrum of the mass analysis obtained from peptide fraction of near the flow path position A12 is shown in FIG. 9.

In FIG. 9, “C” indicates a peak derived from carbonic anhydrase, and “T” indicates a peak derived from trypsin. FIG. 9 also shows both of the results for the A flow path and the B flow path. While it is known from the results of FIG. 7 that carbonic anhydrase is converged in the flow path A near the position, it is understood that the signal of peptide fragment obtained by a tryptic digestion of carbonic anhydrase is also strongly observed from the flow path A. The above-described results show that digestion/fragmentation reactions for carbonic anhydrase by trypsin are progressed in about 15 minutes under the condition for inhibiting the diffusion of peptide fraction. While only one supplying operation of water vapor is illustrated in the present example, a plurality of supplying operations in shorter duration time may alternatively be carried out to ensure the enzyme reaction without disturbing the convergence condition.

While the invention has been particularly shown and described with reference to exemplary embodiments thereof, the invention is no limited to these embodiments. It will be understood by those of ordinary skill in the art that various changes in form and details may be made therein without departing from the spirit and scope the present invention as defined by the claims.

What is claimed is:

1. A method for degrading peptides, comprising:
preparing two or more separated peptide fractions in carriers;
drying said peptide fractions for each of said carriers;
bringing a protease into contact with dried said peptide fractions; and
forming independent liquid membranes of a solvent respectively over surfaces of said peptide fractions contacted with said protease for each of said carriers.

2. The method for degrading peptides as set forth in claim 1, wherein said carrier is a flow path for electrophoresis, and said peptide fraction is separated by migrating through said flow path.

3. The method for degrading peptides as set forth in claim 1, wherein said liquid membrane is formed over the surface of said peptide fraction by generating water vapor.

4. The method for degrading peptides as set forth in claim 1, wherein the formation of said liquid membrane is maintained by containing said peptide fraction within a tightly sealed atmosphere within a chamber, said chamber being fully charged with water vapor and inert gas.

5. The method for degrading peptides as set forth in claim 1, further including evaporating said solvent while supplying an inert gas.

6. The method for degrading peptides as set forth in claim 1, wherein said solvent contains a volatile organic solvent.

7. The method for degrading peptides as set forth in claim 6, wherein said volatile organic solvent contains a basic nitrogen compound.

8. The method for degrading peptides as set forth in claim 1, further including:
preparing a protease solution containing said protease dissolved therein;
applying said protease solution over said peptide fraction; and
drying said peptide fraction applied with said protease solution.

9. The method for degrading peptides as set forth in claim 1, further including:
preparing a protease powder by freeze-drying said protease; and
mixing said peptide fraction in said protease powder.

10. A method for analyzing peptides, wherein a mass analysis of peptide fractions degraded by the method for degrading peptides as set forth in claim 1 is conducted.

11. A device for degrading peptides, comprising:
a sample holding unit for holding a carrier carrying a dried mixed sample, said mixed sample containing two or more separated peptide fractions and a protease, the protease being in contact with said peptide fractions;
a vapor supplying unit for supplying water vapor in said mixed sample; and
a sensor for detecting formations of respectively independent liquid membranes of a solvent over the surfaces of said peptide fractions by said water vapor for each of flow paths, the peptide fraction being in contact with said protease.

12. The device for degrading peptides as set forth in claim 11, wherein said carrier is a flow path of electrophoresis, and said peptide fraction is separated by migrating through said flow path.

13. The device for degrading peptides as set forth in claim 11, further comprising a chamber containing said mixed sample within a tightly sealed atmosphere therein,
wherein said vapor supplying unit supplies said water vapor in said chamber.

14. The device for degrading peptides as set forth in claim 11, further comprising a chamber containing said mixed sample within a tightly sealed atmosphere therein,
wherein said vapor supplying unit supplies an inert gas in said chamber.

15. The device for degrading peptides as set forth in claim 11, further comprising a chamber containing said mixed sample within a tightly sealed atmosphere therein,
wherein said vapor supplying unit supplies said water vapor and an inert gas in said chamber.

16. The device for degrading peptides as set forth in claim 11, wherein said solvent contains a volatile organic solvent.

17. The device for degrading peptides as set forth in claim 11, wherein said volatile organic solvent contains a basic nitrogen compound.

18. A device for analyzing peptides, comprising a mass analysis section for conducting mass analysis of the peptide fraction degraded in the device for degrading peptide as set forth in claim 11.
